(19) United States
(12) Patent Application Publication
(10) Pub. No.: US 2014/0336609 A1
(43) Pub. Date: Nov. 13, 2014

(54) MOISTURE BARRIER FOR CLOTHING

(71) Applicant: SONJA ALBERTSEN, Temecula, CA (US)

(72) Inventor: SONJA ALBERTSEN, Temecula, CA (US)

(21) Appl. No.: 13/889,321

(22) Filed: May 7, 2013

(51) Int. Cl. A61F 13/56

(52) U.S. Cl. CPC A61F 13/5611
USPC 604/385.05; 604/385.03

(57) ABSTRACT

A Moisture Barrier for Clothing includes a pad having an upper layer made from a soft woven material, such as cotton, an intermediate absorbent layer, a moisture barrier layer, and an adhesive layer. The Moisture Barrier for Clothing is oval in shape, accommodating its length and width, depending upon the user's needs. The Moisture Barrier for Clothing is virtually undetectable while used and can be used in conjunction with sheer or tight-fitting clothing, such as sheer summer shorts, and can be worn directly on the woman's pants without the need for underwear or panties. The Moisture Barrier for Clothing is shaped and may be formed with seam-receiving notches to closely adhere to the inside surface of outer clothing, while remaining virtually undetectable, and can be color-matched to the clothing being worn, or to the woman's skin tone, to prevent any see-through aspects of a sheer or thin garment.

### RELATED APPLICATION

This application claims the benefit of priority to U.S. Provisional Application Ser. No. 61/643,452, entitled “Moisture Barrier for Clothing” filed May 7, 2012, and currently co-pending.

### FIELD OF THE INVENTION

The present invention is generally related to protective pads intended to prevent moisture stains, and wear and tear on clothing. The present invention is more particularly, though not exclusively, useful as a moisture barrier to be worn directly on the inside of the outer clothing and to prevent moisture from contacting or being absorbed by the outer clothing.

### BACKGROUND OF THE INVENTION

Vaginal moisture has been, and continues to be, an issue that concerns women. Indeed, for centuries women have sought solutions for catching the flow of the blood during menstruation. Since the 4^{th} century AD, women have been using pads to catch menstrual blood flow, and the technology for these pads has continuously improved. For instance, Benjamin Franklin was thought to have created a wartime wound pad that was later used for protection during menstruation, and the first commercially manufactured disposable menstruation pad was available in 1896 by Johnson & Johnson.

The first menstrual pads consisted of wood pulp bandages and evolved into cloth pads, typically manufactured by the same companies that manufactured bandages. Despite the growing availability of menstrual pads, they were unavailable for most women due to the high cost. As a result, many women continued to use pads fashioned from inexpensive and readily available materials. It took several years for disposable menstrual pads to become commonplace and they are now used throughout most of the industrialized world.

Some of the first disposable pads were generally in the form of a cotton wool rectangle covered with an absorbent liner. The liner ends were extended from the front and back of the pad so as to fit through loops in a special girdle or belt worn beneath the woman's undergarments While it could be effective, this design was notorious for slipping either forward or back of the intended position which often resulted in leakage. Later an adhesive strip was placed on the bottom of the pad so that the pad would attach to the saddle of the panties. This attachment prevented the slippage, and quickly became the preferred pad for women.

The shape, design and materials used to make pads have also changed throughout the years. Earlier materials were not absorbent and effective, and thus early pads were up to two centimeters thick, and leaks were a major problem. To address these challenges, some variations were introduced such as the quilting of the lining, adding flaps, or “wings,” to the side of the pad, and reducing the thickness of the pad by utilizing highly absorbent materials, such as polyacrylate superabsorbent gels derived from petroleum. This results in a highly absorbent, yet oversized pad suitable for absorbing the heavy flow of menstruation. While effective in absorbing menstrual bleeding, menstrual pads are easily visible underneath a woman's clothing, and often dictate the clothing worn in order to minimize the visible presence of the pad. In addition, such menstrual pads need to be worn inside the underwear or panties.

As an alternative to the highly absorbent menstrual pads, a line of thinner pads, commonly referred to as a pantiliner, or panty shield, was developed. Common pantiliners consist of an absorbent piece of material worn in the gusset of a woman's underwear or panties that are suitable for absorbing daily vaginal discharge, light menstrual flow, tampon and menstrual cup backup, spotting, post-intercourse discharge and slight urinary incontinence.

Pantiliners are typically thinner and often narrower than menstrual pads and as a result, they absorb much less liquid than pads. Disposable pantiliners are typically made with an adhesive on the back of the pad to hold them in place in the underwear or panties, and some styles have tabs or “wings” which wrap around the panties, providing additional stability. Pantiliners are effective for addressing concerns from moderate vaginal discharge. However, pantiliners require attachment to panties for placement, and are large enough that they are visible when worn underneath sheer or tight-fitting clothing.

In addition to commonly available menstruation pads and pantiliners, reusable cloth pads are still available. These pads are typically made of cotton (often organic), and can be washed and reused for years. These pads, however, are often thicker, more visible during use, and thus unsuitable for use with sheer or tight-fitting clothing.

In light of the above, it would be advantageous to provide a moisture barrier for clothing to be worn by women that wish to avoid traditional undergarments, such as underwear or panties, while wearing sheer or tight-fitting clothing. It would be advantageous to provide a moisture barrier for clothing to be worn by women that wish to avoid unpleasant underwear bunching up or tightness resulting from wearing undergarments, such as when wearing tight-fitting clothing, but desire to have protection for direct vaginal contact with clothing. It would also be advantageous to provide a moisture barrier for clothing that was virtually undetectable while used so that it could be used in conjunction with thin clothing, such as sheer summer shorts. It would also be advantageous to provide a disposable specific-use moisture barrier for clothing to be worn by women having non-menstruation discharge that can be worn directly on the woman's pants without the need for underwear or panties. It would likewise be advantageous to provide a moisture barrier for clothing that is shaped to closely adhere to the inside surface of outer clothing, such as shorts, while remaining virtually undetectable. It would also be advantageous to provide a moisture barrier for clothing having an adhesive coating on a colored backing that is color-matched to the clothing being worn, or to the woman's skin tone, to prevent any see-through aspects of a sheer or thin garment.

### BRIEF DESCRIPTION OF THE DRAWING

The nature, objects, and advantages of the present invention will become more apparent to those skilled in the art after considering the following detailed description in connection with the accompanying drawings, in which like reference numerals designate like parts throughout, and wherein:

FIG. 1 is a diagrammatic view of a woman wearing shorts having an inseam and made from sheer materials, and equipped with a preferred embodiment of the Moisture Barrier for Clothing of the present invention positioned in the crotch region of the inseam;

FIG. 2 is a cross-sectional view of the clothing of FIG. 1 as taken along line 2-2 of FIG. 1, showing the placement of the Moisture Barrier for Clothing of the present invention in the crotch region of the shorts and covering a centralized portion of the shorts adjacent to the woman's vagina;

FIG. 3 is a cross-sectional view of the clothing of FIGS. 1 and 2 as taken along line 3-3 of FIG. 2, showing the placement of the Moisture Barrier for Clothing of the present invention in the crotch region of the shorts along the inseam to provide a moisture barrier between the woman's vagina and the clothing;

FIG. 4 is a top plan view of a preferred embodiment of the Moisture Barrier for Clothing of the present invention showing its overall shape, curvature, and a woven top layer;

FIG. 5 is a cross-sectional view of the preferred embodiment of the Moisture Barrier for Clothing of the present invention as taken along line 5-5 of FIG. 4, showing a pad having a top layer, one or more intermediate layers having absorbent qualities, a moisture barrier layer having an outer adhesive layer covered by a removable strip;

FIG. 6 is a top plan view of an alternative embodiment of the Moisture Barrier for Clothing of the present invention showing a number of peripheral notches formed in the periphery of the pad to facilitate the smooth and accurate placement of the Moisture Barrier for Clothing in the crotch area of a woman's pants; and

FIG. 7 is a top plan view of another alternative embodiment of the Moisture Barrier for Clothing of the present invention in a streamlined shape, showing a number of notches formed in the periphery of the pad to facilitate the smooth and accurate placement of the Moisture Barrier for Clothing in the crotch area of a woman's pants.

### DETAILED DESCRIPTION OF A PREFERRED EMBODIMENT

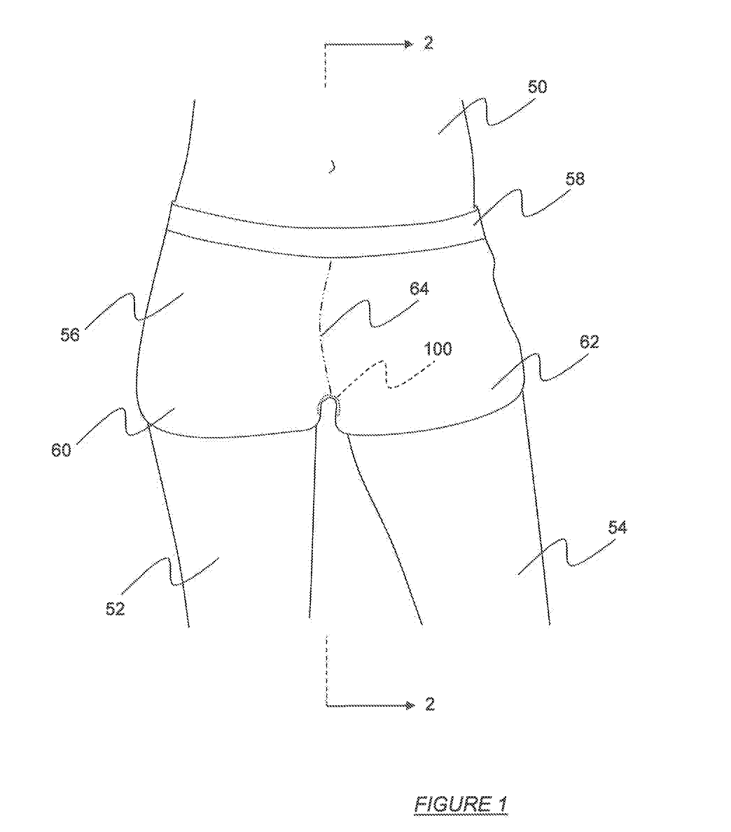

Referring initially to FIG. 1, a diagrammatic view of a preferred embodiment of the Moisture Barrier for Clothing of the present invention is depicted and generally designated 100. In FIG. 1, a woman 50 wearing shorts 56 made from sheer materials and having a waistband 58 and an inseam 64 is shown. Shorts 56 are equipped with the preferred embodiment 100 of the Moisture Barrier for Clothing of the present invention (shown in dashed lines) positioned in the crotch region of the inseam 64. From FIG. 1, it can be seen that the Moisture Barrier for Clothing 100 of the present invention does not extend much beyond the inseam 64 region of the shorts 56 and does not extend to contact legs 52 or 54, nor does it extend much into the leg portions 60 and 62 of the shorts 56. Consequently, the existence of the Moisture Barrier for Clothing 100 of the present invention in the shorts is undetectable.

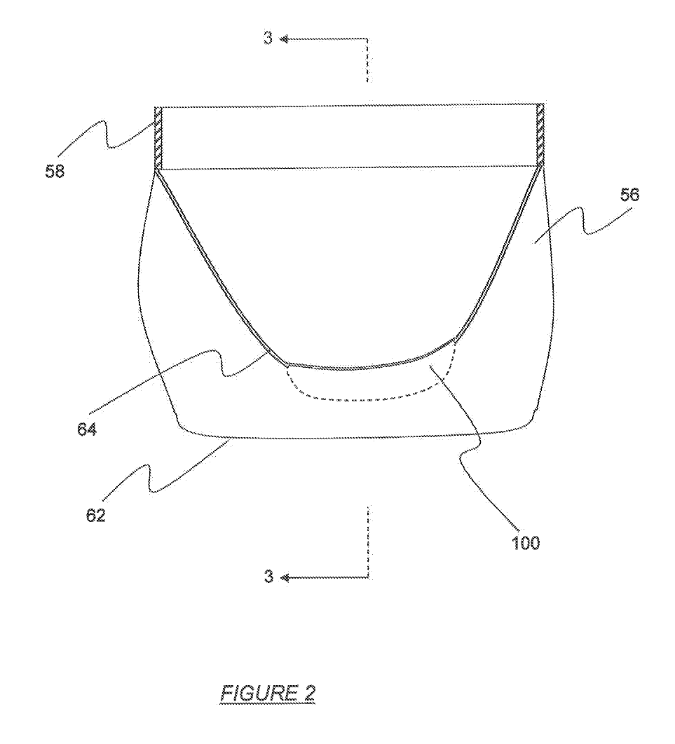

FIG. 2 shows a cross-sectional vie of the shorts 56 of FIG. 1 as taken along line 2-2 of FIG. 1. From this view, the placement of a preferred embodiment 100 of the Moisture Barrier for Clothing of the present invention over inseam 64 and adjacent the crotch region of the shorts 56 is clearly appreciated. In this position, the Moisture Barrier for Clothing 100 of the present invention covers a centralized portion of the shorts 56 adjacent to the woman's vagina (not shown). It is to be appreciated that the precise placement of the Moisture Barrier for Clothing 100 of the present invention will depend upon the particular anatomy of the woman 50 and can be moved forward or backward to provide for the most comfort and the most effective barrier to moisture reaching a garment.

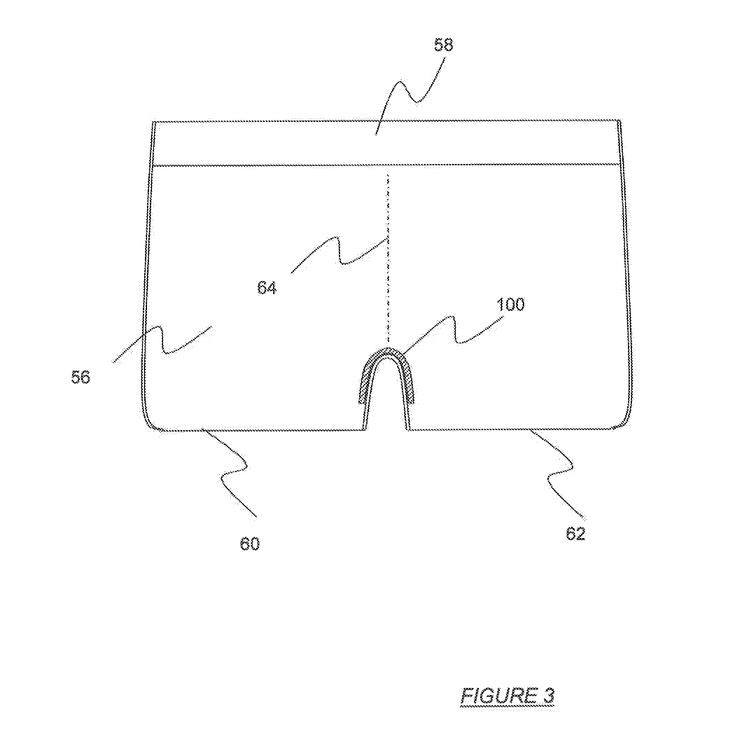

Referring now to FIG. 3, a cross-sectional view of the clothing of FIGS. 1 and 2 as taken along line 3-3 of FIG. 2 is shown. The Moisture Barrier for Clothing of the present invention is placed in the crotch region of the shorts 56, along the inseam 64. As shown, the Moisture Barrier for Clothing 100 is conformed and in position directly adjacent to the inseam 64 of shorts 56 to provide a moisture barrier between the woman's vagina (not shown) and the clothing.

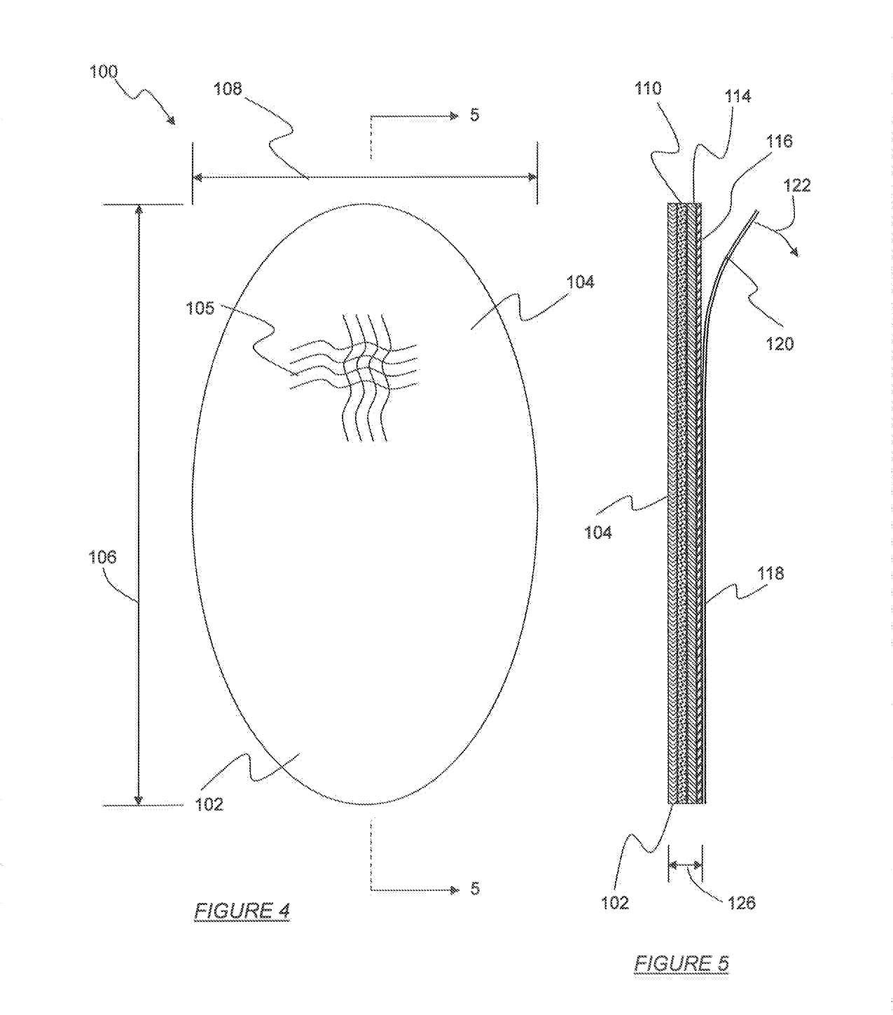

Referring now to FIG. 4, a top plan view of a preferred embodiment 100 of the Moisture Barrier for Clothing of the present invention is shown. From this top plan view, the overall shape and curvature of the Moisture Barrier for Clothing 100 of the present invention is clearly appreciated. The Moisture Barrier for Clothing 100 includes a pad 102 having an upper layer 104 that is made from a soft woven material, such as cotton, as illustrated in lines 105 representing the woven material.

In a preferred embodiment, of the Moisture Barrier for Clothing of the present invention is oval in shape, and has a length 106 of approximately 6 inches, and a width 108 of approximately 3 inches. It is to be appreciated, and fully contemplated herein, that the oval shape, and the length 106 and width 108 are merely exemplary of a preferred embodiment, and that variations are contemplated without departing from the present invention. In a preferred embodiment, the length 206 to width 108 ratio equals or exceeds 2.

FIG. 5 is a cross-sectional view of the preferred embodiment 100 of the Moisture Barrier for Clothing of the present invention as taken along line 5-5 of FIG. 4. The various layers within the preferred embodiment 100 of the Moisture Barrier for Clothing of the present invention are shown, and include an upper layer 104 made from a soft woven material, as mentioned above. Since this upper layer 104 contacts the genital area of the woman, the materials of the upper layer 104 can be hypo-allergenic, acid free, and materials known in the art to be well tolerated in such skin contact applications. in a preferred embodiment, organic materials, including, but not limited to cotton, would be considered beneficial. Alternatively, soft non-woven materials, including, but not limited to non-woven polypropylene, can also be used.

A first intermediate layer 110 is adjacent to the pad 102, and includes an absorbent material. In a preferred embodiment, the intermediate layer 110 may include a sheet formed to include moisture absorbing materials, such as desiccants or gels. One such moisture absorbent material for the intermediate layer 110 may consist of polyacrylate superabsorbent gels that are well known in the industry for the qualities for absorbing and retaining moisture. These polyacrylate superabsorbent gels are known to be relatively inexpensive and these gels may be impregnated into a natural or synthetic non-woven material that allows moisture to pass through the material where it is captured by the absorbent gels.

Adjacent to the intermediate layer 110 is a moisture barrier layer 114 that prevents moisture from passing from the upper layer 104, through the absorbent intermediate layer 110, to a garment, such as shorts 56 in FIGS. 1-3. In the preferred embodiment, the moisture barrier layer 114 can be made of a soft polymer material that prevents passage of moisture. The soft polymer material used for the moisture barrier layer 114 may be made from polyethylene films that are well known in the art for the flexibility and moisture barrier qualities.

The surface of the moisture barrier layer 114 opposite the absorbent intermediate layer 110 is coated with an adhesive layer 116 that is covered with a peel-off covering 118. As shown, a portion 120 of peel-off covering 118 may be removed in direction 122 to reveal the adhesive layer 116 for placement within the garment being worn (as shown in FIG. 1). As shown in FIG. 5, the entire surface of the moisture barrier layer 114 may be coated with adhesive layer 116. However, it is to be appreciated that the adhesive layer 116 may be applied to only a small portion of the moisture barrier layer 114 sufficient to maintain proper positioning along the inseam 64 (shown in FIGS. 1-3) of the garment, such as shorts 56.

With reference to FIG. 1, it can be appreciated that when Moisture Barrier for Clothing 100 of the present invention is used, the adhesive layer 116 contacts the garment, such as shorts 56. Thus, the only portion of the Moisture Barrier for Clothing 100 of the present invention that is away from the body and might be visible through the garment 56 is the adhesive layer 116. In embodiments where the adhesive layer is not opaque (e.g., clear or translucent), the only portion visible through the garment will be the moisture barrier 114.

In order to most effectively conceal the existence of the Moisture Barrier for Clothing 100, an opaque adhesive layer 116 may be patterned or color-matched to the clothing being worn, or to the woman's skin tone, to prevent any see-through aspects of a sheer or thin garment. Also, where the Moisture Barrier for Clothing 100 includes a partial or non-opaque adhesive layer 116, the moisture barrier layer 114 may also be patterned or color-matched to the clothing being worn, or to the woman's skin tone. By color matching any visible portion of the Moisture Barrier for Clothing 100 to the clothing or to the woman's skin tone, the Moisture Barrier for Clothing 100 of the present invention can be used undetected.

It is contemplated that a variety of Moisture Barrier for Clothing 100 of the present invention are provided with a variety of clothing and skin tone matching colors. By selecting the Moisture Barrier for Clothing 100 with the appropriate clothing color or skin tone, the woman may use the Moisture Barrier for Clothing 100 without any undergarments, and remain undetected.

Moisture Barrier for Clothing 100 of the present invention is manufactured to have a thickness 126 which, in a preferred embodiment, is less than 0.0625 inches (one-sixteenth of an inch). This provides for the Moisture Barrier for Clothing 100 that is extremely thin, flexible and conforms closely to the garment.

In addition to the features mentioned above, layers 102, 104, 110, and 114 may be formed from a material having an elastic characteristic that provides for stretching in both length 106 and width 108. This elasticity allows for a wearer to carefully conform the Moisture Barrier for Clothing 100 of the present invention to the garment. Such a precise placement and gap-free adhesion to the garment eliminates any signs that the Moisture Barrier for Clothing 100 is being worn.

The Moisture Barrier for Clothing 100 of the present invention, by design, is thin, absorptive, flexible, moderately stretchable, can be used with virtually any pant-type garment, and its use is virtually undetectable. This is in stark contrast to the bulky pads currently in existence, and the unwanted visible panty lines that plague wearers of sheer or tight-fitting clothing. By using the Moisture Barrier for Clothing 100 of the present invention, a woman may wear her sheer and/or tight-fitting clothing without underwear, and without the fear that ordinary vaginal moisture will in any way reach the outer garment resulting in undesired discoloration or wetness.

### Alternative Embodiments

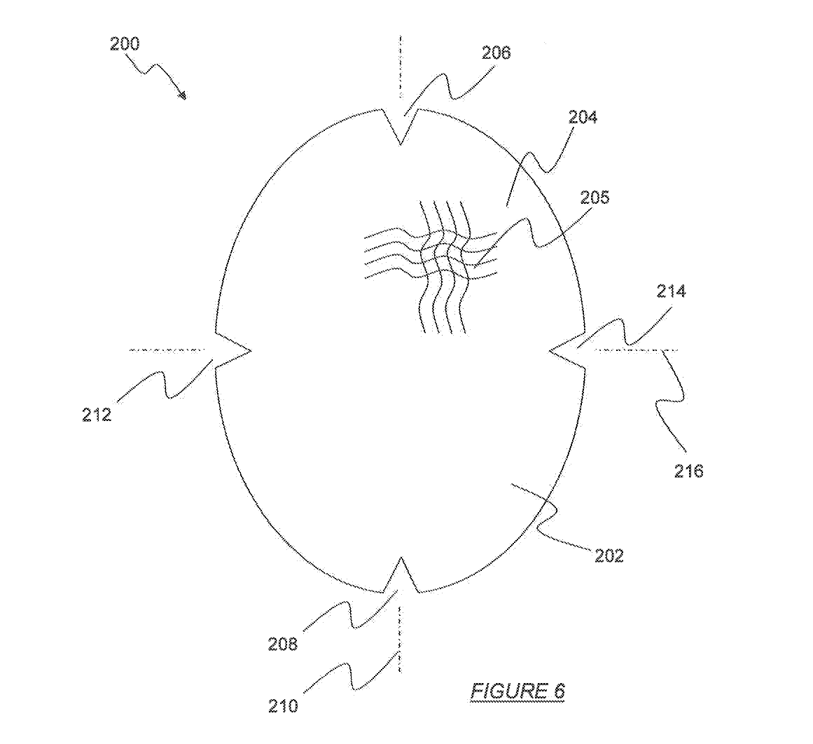

Referring now to FIG. 6, a top plan view of an alternative embodiment of the Moisture Barrier for Clothing of the present invention is shown and generally designated 200. Moisture Barrier for Clothing 200 includes a pad 202 that includes an upper layer 204 that is made from a natural or synthetic material that is suitable for contacting the female genitals and allow for the transmission of moisture. Lines 205 depict a soft woven material from which the upper layer 204 is made.

The remaining layers of the Moisture Barrier for Clothing 200 are similar to those layers described in conjunction with the above described preferred embodiment 100 of the present invention.

Moisture Barrier for Clothing 200 is formed with a number of notches formed in the periphery of the pad to facilitate the smooth and accurate placement of the Moisture Barrier for Clothing 200 in the crotch area of a woman's pants. More specifically, notches 206 and 208 are formed in pad 202 and positioned along axis 210. These notches 206 and 208 facilitate the placement of the Moisture Barrier for Clothing 200 along the inseam 64 of the shorts 56 (shown in FIGS. 1-3) and can accommodate an inseam curvature. Similarly, notches 212 and 214 are formed in pad 202 and positioned along axis 216. These notches 212 and 214 facilitate the placement of the Moisture Barrier for Clothing 200 in shorts 56 having varying shaped leg portions.

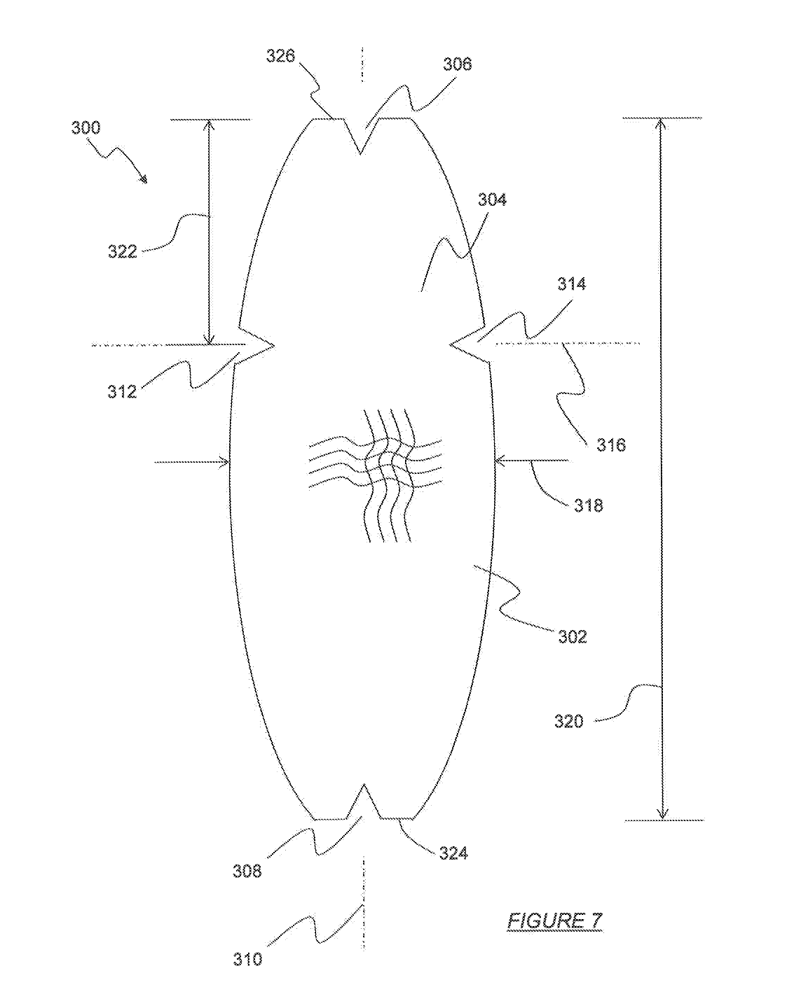

In FIG. 7, a top plan view of another alternative embodiment of the Moisture Barrier for Clothing of the present invention is shown and generally designated 300. The alternative embodiment 300 of the Moisture Barrier for Clothing of the present invention is streamlined in its shape, and has a length 320 of approximately 6 inches, and a width 318 of 2.75 inches at its maximum. In a preferred embodiment, the length to width ratio exceeds 2, but may be greater or lesser than 2 to accommodate different clothing applications.

Moisture Barrier for Clothing 300 includes a pad 302 that includes an upper layer 304 that is made from a natural or synthetic material that is suitable for contacting the female genitals and allow for the transmission of moisture. Lines 305 depict a soft woven material which the upper layer 304 is made from. The remaining layers of the Moisture Barrier for Clothing 300 are similar to those layers described in conjunction with the preferred embodiment 100, and the alternative embodiment 200 of the present invention.

Similar to the notches adopted in the alternative embodiment 200 of the present invention, Moisture Barrier for Clothing 300 is formed with a number of notches formed in the periphery of the pad to facilitate the smooth and accurate placement of the Moisture Barrier for Clothing 300 in the crotch area of a woman's pants. Specifically, notches 306 and 308 are formed in pad 302 and positioned along long axis 310. These notches 306 and 308 facilitate the placement of the Moisture Barrier for Clothing 300 along the inseam 64 of the shorts 56 (shown in FIGS. 1-3) and can accommodate an inseam curvature. Similarly, notches 312 and 314 are formed in pad 302 and positioned along short axis 316. These notches 312 and 314 facilitate the placement of the Moisture Barrier for Clothing 300 in shorts 56 having varying shaped leg portions. In the alternative embodiment 300 of the present invention, the axis 316 for notches 312 and 314 is placed 1.75 inches from the top notch 306, as a distance 322 depicts. In a preferred embodiment, notches 312 and 314 are formed a distance 322 along axis 310 of the length 320, wherein the length 320 to distance 322 ratio exceeds two. In alternative embodiments, the length 320 to distance 322 ratio exceeds three.

The placement of notches 312 and 314 along axis 316 is merely exemplary of a preferred embodiment of the present invention. The distance 322 from end 326 may vary depending on the particular garment being protected, the particular position a wearer prefers or needs protection from moisture transmission, and the preferences or basic anatomy of the wearer.

Notches 306, 308, 312 and 314 are placed along the periphery of the moisture barrier for clothing 300 of the present invention to allow for the placement of the device smoothly on the inside surface of the garment being worn. Typical seams, such as inseam 64 (shown in FIG. 1) represent a slightly thicker portion of the garment, and by forming notches 306, 308, 312 and 314 in the device, the flat end portions 324 and 326 attach securely and smoothly to the inside surface of the garment.

While there have been shown what are presently considered to be preferred embodiments of the present invention, it will be apparent to those skilled in the art that various changes and modifications can be made herein without departing from the scope and spirit of the invention.

What is claimed is:

1. A moisture barrier for clothing, comprising:
a pad having an upper layer;
one or more intermediate absorbent layers adjacent said pad and opposite said upper layer;
a moisture barrier layer adjacent said intermediate absorbent layer; and
an adhesive layer on at least a portion of said moisture barrier layer opposite said absorbent layer.

2. The moisture barrier for clothing of claim 1, wherein said pad further comprises an upper layer that is hypo-allergenic

3. The moisture barrier for clothing of claim 1, wherein said pad further comprises an upper layer that is acid free.

4. The moisture barrier for clothing of claim 1, wherein said intermediate layer comprises absorbent material to retain moisture.

5. The moisture barrier for clothing of claim 1, wherein said adhesive layer is covered with a peel-gaff covering removable to reveal said adhesive layer for placement of said moisture barrier for clothing within the garment being worn.

6. The moisture barrier for clothing of claim 1, wherein said adhesive layer is formed by coating said moisture barrier layer.

7. The moisture barrier for clothing of claim 1, wherein said adhesive layer covers the entirety of said moisture barrier layer.

8. The moisture barrier for clothing of claim 1, wherein said adhesive layer covers a portion of said moisture barrier layer.

9. The moisture barrier for clothing of claim 1, wherein said adhesive layer is selected from the group consisting of a partially opaque adhesive layer, a non-opaque adhesive layer, a patterned adhesive layer, and a color-matched adhesive layer.

10. The moisture barrier for clothing of claim 9, wherein said color matched adhesive layer corresponds to a skin tone.

11. The moisture barrier for clothing of claim 1, wherein said moisture barrier layer is selected from the group consisting of a patterned layer, a layer color-matched to the clothing being worn, and a layer color-matched to the woman's skin tone.

12. The moisture barrier for clothing of claim 1, further comprising;
A long axis and a short axis;
A pair of peripheral notches on said long axis.

13. The moisture barrier for clothing of claim 1, further comprising:
A long axis and a short axis; and
A pair of peripheral notches on said long axis.

14. The moisture barrier for clothing of claim 1, further comprising:
a long axis defining a length and a short axis defining a width; and
a pair of peripheral notches on said long axis comprising a top notch and a bottom notch; and
a pair of peripheral notches on said short axis.

15. The moisture barrier for clothing of claim 14, wherein said peripheral notches on said short axis are formed a distance from said top notch, and wherein the ratio of said length to said distance exceeds two.

16. The moisture barrier for clothing of claim 14, wherein said peripheral notches on said short axis are formed a distance from said top notch, and wherein the ratio of said length to said distance exceeds three.

17. The moisture barrier for clothing of claim 14, wherein said notches facilitate the placement of the moisture barrier for clothing along the inseam of the clothing on the inside surface of the clothing being worn.

18. The moisture barrier for clothing of claim 1, wherein said fiat ends attach securely to the inside surface of the clothing being worn.

19. The moisture barrier for clothing of claim 1, wherein said pad has a length and a width, and a ratio of said length to said width of at least 2.

20. The moisture barrier for clothing of claim 1, wherein said pad has a length with a long axis and a width with a short axis, and a ratio of said length to said width of at least 2.
